(19) United States
(12) Patent Application Publication
(10) Pub. No.: US 2008/0267832 A1
(43) Pub. Date: Oct. 30, 2008

(54) Aroma porcelain diffuser

(75) Inventor: Vicki Chen, Shanghai (CN)

Correspondence Address: TROXELL LAW OFFICE PLLC, SUITE 1404, 5205 LEESBURG PIKE, FALLS CHURCH, VA, 22041, US

(73) Assignee: Top Harvest Design & Manufacture Co., Ltd.

(21) Appl. No.: 11/790,469

(22) Filed: Apr. 25, 2007

(51) Int. Cl. A61L 9/04

(52) U.S. Cl. 422/123

(57) ABSTRACT

An aroma porcelain diffuser comprises a container seat equipped with a grooved hole and a ringed guide surface for the accommodation of aromatic agent therein, and a microelement breathable and permeable body having a water-permeable and air-ventilating special porcelain wicking head with fine pores densely and evenly formed thereon. The microelement breathable and permeable body also has a suction end with water-permeable and air-ventilating fine pores thereon to mount into the container seat and precisely soak into the aromatic agent retained in the container seat. Via each fine porous cell of the microelement breathable and permeable body, the aromatic agent absorbed by the suction end is transported to contact with the atmosphere outside and naturally generate physical changes thereby. Therefore, the porcelain body thereof works as a medium through which the fragrant scent of the aromatic agent is efficiently released and diffused long and wide in the surrounding environment.

### BACKGROUND OF THE INVENTION

The present invention relates to an aroma porcelain diffuser, comprising a container seat for the accommodation of aromatic agent, and a microelement breathable and permeable body made up of a water-permeable and air-ventilating special porcelain wicking head with fine pores densely and evenly formed thereon, and a suction end wherein the microelement breathable and permeable body with a porous property can absorb a great quantity of the aromatic agent and release the fragrant scent which, via each fine porous cells of the porcelain wicking head, is effectively and evenly diffused in the surrounding area in a long-lasting and continuous manner.

Conventional aroma diffusers applied to incense or refresh the indoor air include aromatherapy candles, essential oil burners, or press-type aroma-emitting devices, etc. In application, these conventional aroma diffusers must be lighted up for burning or operated via spraying movement so that aroma agents can be released to interact with the air circulation and become airborne. In these ways, the fragrant scents can be emitted to diffuse in the surrounding area of an indoor space thereof. However, the press-type aroma-emitting devices must be manually operated so that the stream of the aroma agent can be ejected outwards to contact the atmosphere and generate fragrant scent thereby. When the fragrant odor evaporates and dies out, the aroma-emitting devices thereof must be pressed again to transport the aroma agent for use so that the indoor air can be kept fresh and fragrant again. Thus, in addition to the inconvenience of repeated pressing in application, the conventional press-type aroma-emitting devices fail to diffuse the fragrant scents of aromatic agent in a long-lasting and continuous manner. On the other hand, the burning-type aroma diffusers like aromatherapy candles and the essential oil burners can keep long-lasting fragrant smell. Even though, the aromatic scent is generated via the method of burning which, in addition to the risk of firing, can also produce dangerous carbonized odor and pose a great danger to people's health. Besides, when encountering impact from outside, the burning-type aroma diffusers above can easily get turned over or broken and lead to the danger of fire, which makes the conventional burning-type aroma diffusers quite dangerous in application.

Furthermore, conventional porcelain aromatic diffusers utilize the constituents, including CaO (30.4%), MgO (21.9%), CO₂ (47.7%) which belong to high melting-point porcelain clay and are not equipped with the benefits to absorb aromatic agent and release the fragrant scent thereof in application.

### SUMMARY OF THE PRESENT INVENTION

It is, therefore, the primary purpose of the present invention to provide an aroma porcelain diffuser wherein a microelement breathable and permeable body having fine pores evenly and densely formed on the interior and the external surface thereon is equipped with a porous property to absorb a great quantity of essential oils and release the fragrant scents as well; whereby the microelement breathable and permeable body can absorb aromatic agent on a complete scale to increase the contact area with the atmosphere and maintain long-lasting and continuous fragrant odor in the air without the inconvenience of the aforementioned conventional press-type or burning-type aroma diffusers, achieving effective diffusion of aromatic scents in a better and more convenient way. Besides, depending on the indoor space or location, a fragrant porcelain container seat of various sizes can be flexibly chosen to match to the microelement breathable and permeable body, achieving esthetic beauty as well as practical benefits thereby.

It is, therefore, the second purpose of the present invention to provide an aroma porcelain diffuser wherein the microelement breathable and permeable body is molded into a water-permeable and air-ventilating special porcelain wicking head with fine pores densely and evenly formed thereon, and a suction end to absorb the aromatic agent; whereby via each fine porous cell of the porcelain wicking head of the microelement breathable and permeable body, the aromatic agent absorbed is transported to contact with the atmosphere outside and naturally generate physical changes thereby; therefore, the porcelain body thereof works as a medium through which the fragrant scent of the aromatic agent is efficiently released and diffused evenly in the surrounding environment, achieving long-lasting and continuous diffusion of the fragrant scents in the air till the aromatic agent is used up.

### BRIEF DESCRIPTION OF THE DRAWINGS

FIG. 1 is an exploded perspective view of the present invention.

FIG. 2 is an assembled perspective view of the present invention.

FIG. 3 is an assembled cross sectional view of the present invention.

FIG. 4 is a diagram showing the present invention in application.

### DETAILED DESCRIPTION OF THE PREFERRED EMBODIMENTS

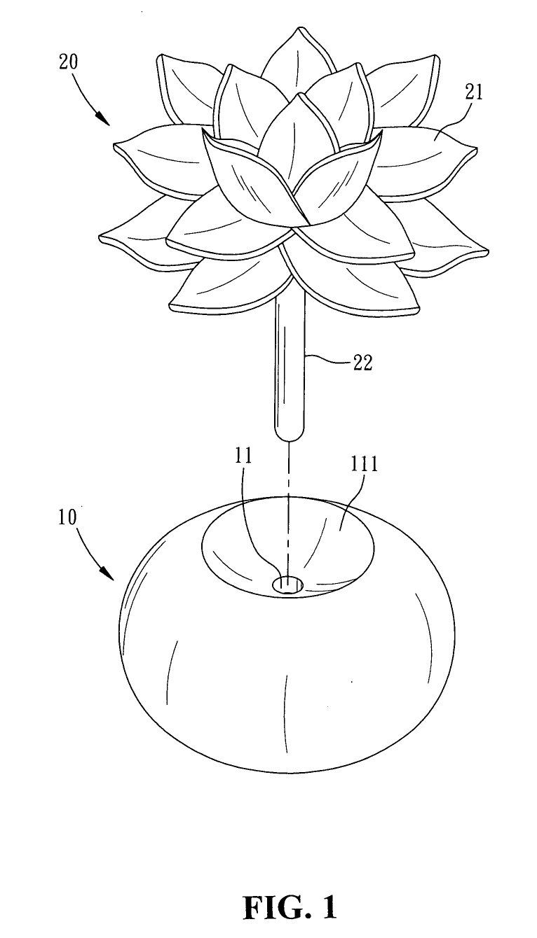

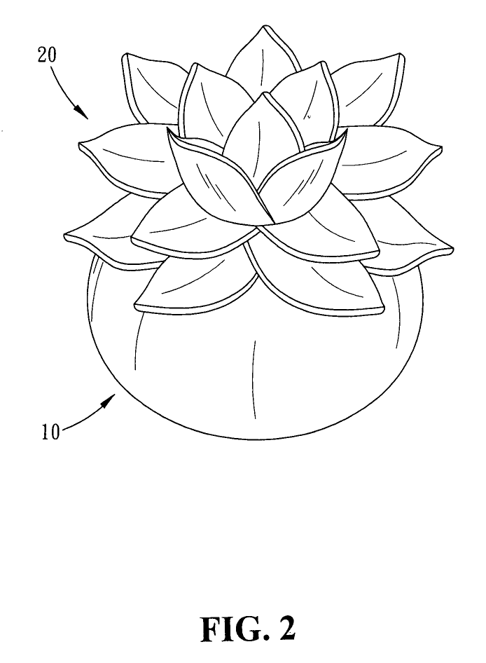

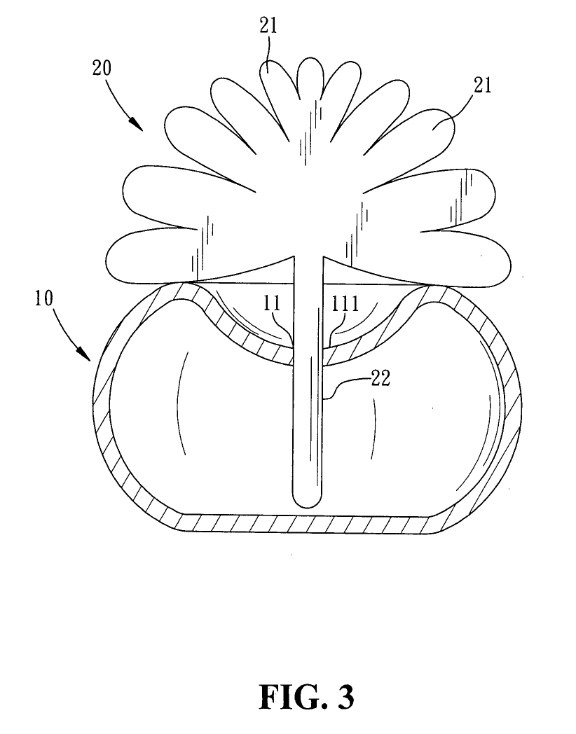

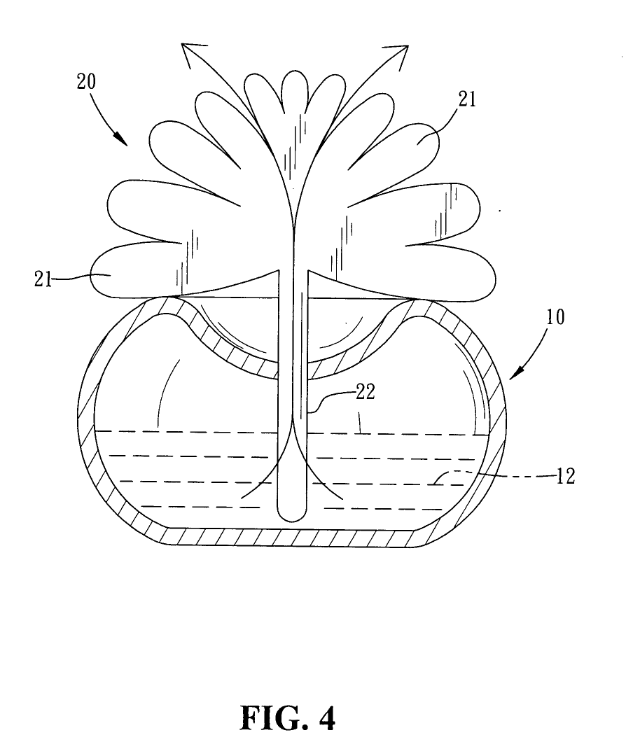

Please refer to FIG. 1 showing an exploded perspective view of the present invention (accompanied by FIGS. 2 through 4 inclusive). The present invention relates to an aroma porcelain diffuser, comprising a container seat 10 and a microelement breathable and permeable body 20 wherein the container seat 10 is equipped with a grooved hole 11 defined by a ringed guide surface 111 thereon for the accommodation of aromatic agent 12 (various type of fragrances such as essential oils) therein, and the container seat 10 can be molded into various stylistic shapes. The microelement breathable and permeable body 20, composed of SiO₂ (35.96%), Al₂O₃ (59.5%), Fe₂O₃ (0.1%), TiO₂ (0.04%), CaO (0.25%), MgO (0.02%), K₂O (0.82%), Na₂O (0.7%), Lg loss (2.61%), is molded into a liquid-permeable and air-ventilating special porcelain wicking head 21 each having fine pores evenly and densely formed on the interior and the external surface thereon. The microelement breathable and permeable body 20 can also be shaped into various types of objects to match to the ringed guide surface 111 of the container seat 10 and offer a unique and stylistic decoration effect thereby. In addition, the microelement breathable and permeable body 20 also includes a suction end 22 extending at one end that, equipped with water-permeable and air-ventilating fine pores thereon, is guided through the grooved hole 11 to mount into the container seat 10, permitting one end of the suction end 22 to precisely extend and soak into the aromatic agent 12 retained in the container seat 10 therein. Meanwhile, the suction end 22 is utilized to absorb and suck up the aromatic agent 12 that, via each fine porous cell of the microelement breathable and permeable body 20, is transported outwards to contact with the atmosphere outside and naturally generate physical changes thereby. Thus, the porcelain body thereof works as a medium through which the fragrant scent of the aromatic agent 12 is efficiently released and evenly diffused in the surrounding environment. Via air circulation, the fragrant scent of the aromatic agent 12 emitted can become airborne, and the fragrant odor in the air can be maintained in a long-lasting and continuous manner till the aromatic agent 12 is used up. Furthermore, the microelement breathable and permeable body 20 with fine air pores evenly and densely formed on the interior and the external surface thereon is equipped with an absorbing property to suck up a great quantity of essential oils and release the fragrant scents thereof as well. Therefore, the microelement breathable and permeable body 20 can absorb the aromatic agent 12 on a complete scale to increase the contact area with the atmosphere and maintain long-lasting and continuous fragrant smell in the air without the inconvenience of the conventional aromatic diffusers utilizing the method of pressing or burning, etc., providing a better and more efficient aroma porcelain diffuser with the benefits of security, practicality, and esthetic beauty.

Moreover, the microelement breathable and permeable body 20 is composed of SiO₂ (35.96%), Al₂O₃ (59.5%), Fe₂O₃ (0.1%), TiO₂ (0.04%), CaO (0.25%), MgO (0.02%), K₂O (0.82%), Na₂O (0.7%), Lg loss (2.61%) and molded into a liquid-permeable and air-ventilating special porcelain wicking head 21 with fine pores evenly and densely formed on the interior and the external surface thereon. High melting-point aluminum oxide is utilized as solid-phase raw material, while lower melting-point porcelain clay is used as liquid-phase material. In the first stage of the sintering process, when the powder particles reach the contact point and touch each other, the neck region at the junction between the powder particles will grow to about 20% of the grain radius, which is called the initial sintering. During this stage, the shrinkage percentage is no more than 4-5%. In the middle stage thereof, the grain boundaries will expand and the neck growth will enable the movement of the grain boundaries, resulting in the deformation of the crystal grain growth, and making the air pores at the crystal boundaries arrange into consecutive tubular shapes and form a network thereon. During this stage, the shrinkage percentage is about 5-20%, and the density also reaches 95%. Besides, the liquid-phase material thereof is added to make the clay body equipped with certain strength thereby, and the raw material with the fine air pores formed on the crystal boundaries is also utilized to provide a porous property for the absorption of essential oils and the release of the fragrant scents thereby.

What is claimed is:

1. An aroma porcelain diffuser, comprising a container seat and a microelement breathable and permeable body wherein the container seat is equipped with a grooved hole defined by a ringed guide surface for the accommodation of aromatic agent therein, and the microelement breathable and permeable body is made up of a water-permeable and air-ventilating special porcelain wicking head each having fine pores evenly and densely formed on the interior and external surface thereon; in addition, the microelement breathable and permeable body also has a suction end extending at one end that, having water-permeable and air-ventilating fine pores formed thereon, is guided through the grooved hole to mount into the container seat, permitting one end of the suction end to precisely extend and soak into the aromatic agent retained in the container seat therein; meanwhile, the suction end is utilized to absorb and suck up the aromatic agent that, via each fine porous cell of the microelement breathable and permeable body, is transported outwards to contact with the atmosphere outside and naturally generate physical changes thereby;
therefore, the porcelain body thereof works as a medium through which the fragrant scent of the aromatic agent is efficiently released and evenly diffused in the surrounding environment, providing a better and more efficient aroma porcelain diffuser with the benefits of security, practicality, and esthetic beauty.

2. The aroma porcelain diffuser as claimed in claim 1 wherein The microelement breathable and permeable body, molded into the special porcelain wicking head, is composed of SiO₂ (35.96%), Al₂O₃ (59.5%), Fe₂O₃ (0.1%), TiO₂ (0.04%), CaO (0.25%), MgO (0.02%), K₂O (0.82%), Na₂O (0.7%), Lg loss (2.61%).

3. The aroma porcelain diffuser as claimed in claim 1 wherein the container seat can be flexibly molded into various stylistic shapes in appearance.

4. The aroma porcelain diffuser as claimed in claim 1 wherein the microelement breathable and permeable body can also be shaped into various types of objects to reciprocally combine with the container seat and offer a unique and stylistic decoration effect thereby.

5. An aroma porcelain diffuser wherein the porcelain aroma porcelain diffuser is composed of SiO₂ (35.96%), Al₂O₃ (59.5%), Fe₂O₃ (0.1%), TiO₂ (0.04%), CaO (0.25%), MgO (0.02%), K₂O (0.82%), Na₂O (0.7%), Lg loss (2.61%), and high melting-point aluminum oxide is utilized as solid-phase raw material while lower melting-point porcelain clay is used as liquid-phase material, which are molded into a microelement breathable and permeable body with porcelain wicking head disposed thereon; in the first stage of the sintering process, when the powder particles reach the contact point and touch each other, the neck region at the junction between the powder particles will grow to about 20% of the grain radius, which is called the initial sintering; during this stage, the shrinkage percentage is no more than 4-5%; in the middle stage thereof, the grain boundaries will expand and the neck growth will enable the movement of the grain boundaries, resulting in the deformation of the crystal grain growth, and making the air pores at the crystal boundaries arrange into consecutive tubular shapes and form a network thereon; during this stage, the shrinkage percentage is about 5-20%, and the density also reaches 95%; besides, the liquid-phase material thereof is added to make the clay body equipped with certain strength thereby, and the raw material with the fine air pores formed on the crystal boundaries is also utilized to provide a porous property for the suction of essential oils and the release of the fragrant scents thereby.
